(19) United States
(12) Patent Application Publication
(10) Pub. No.: US 2021/0213248 A1
(43) Pub. Date: Jul. 15, 2021

(54) Umbrella Catheter Device

(71) Applicant: Rishi Razdan, M.D., Jacksonville, FL (US)

(72) Inventor: Rishi Razdan, M.D., Jacksonville, FL (US)

(21) Appl. No.: 16/738,417

(22) Filed: Jan. 9, 2020

(51) Int. Cl. A61M 25/00

(52) U.S. Cl. CPC A61M 25/0074; A61M 2205/0266; A61M 2025/0079

(57) ABSTRACT

A catheter device having a hollow tube with a proximal end and a distal end. The device also having a linkage inside the hollow tube extending between the proximal end and the distal end of the hollow tube and a plurality of ribs connected to a first connection point at the distal end of the hollow tube. The ribs have a first end at the first connection point and a second end opposite the first end. Between each rib is a mesh extending from each rib to a different rib of the plurality of ribs. A first handle is at the proximal end of the hollow tube and a second handle is adjacent to the first handle and attached to the linkage. The second handle actuates the plurality of ribs from a retracted position to an extended position.

### TECHNICAL FIELD

The disclosure herein relates to devices used to clear obstructions and objects in veins, arteries and any other tubular structure within the body. Obstructions can form in arteries and veins when a catheter is inserted for permanent or semi-permanent treatments. Specifically, fibrin sheaths or thrombus can form around the outer surface of a hemodialysis catheter, reducing or even completely blocking flow through either lumen of the catheter. The disclosed device relates to a catheter for removing fibrin sheaths, embolisms, or any other objects inside of an artery, vein, or other structure with a lumen. The disclosed device relates to devices that expand within a vein, artery, or bodily structure with lumen while still allowing flow through said passage and capturing the material being removed.

### BACKGROUND

Systems known in the art allow a surgeon to remove objects from a passage within a body. These systems can include netting made of wires, the netting expanding and contracting to form different shapes. The netting can be made of materials that expand, contract, and bend in ways that allow the net to form different shapes within a passage. Such devices require more than one hand to open and close to capture objects, limiting the tasks a user can perform while actuating the device.

It is important for devices in the field to be uniquely suited for the passage or body they are used in due to the delicate nature of the tissues these devices are used near and within. Devices also must be simple to use with one hand. There is a wide variety of procedures that are carried out in veins and arteries, each with their own requirement for stiffness of the wire net, controllability of the expansion and contraction of the wire net, visibility of the wire net on imaging machines, the device's ability to remove or install an object, and to effectively handle and manipulate the size, shape, and consistency of the object in the passage. Applicant addresses problems with devices in the prior art by providing a device that can be actuated with one hand while a user's other hand is used to guide the device through a passage, or perform other functions simultaneously with actuation.

### SUMMARY

The needs set forth herein as well as further and other needs and advantages are addressed by the present embodiments, which illustrate solutions and advantages described below.

There is a need for a device that can be inserted into a vein, artery, or other passage in a body and be expanded in an umbrella shape to capture an object in the body passage and allow a surgeon to withdraw the object with the device.

There is a need for a catheter device that is small enough in cross sectional diameter to be inserted into a body passage and long enough to reach an object or obstruction. There is also a need for the device to have a linkage extending from a proximal end of the device to the distal end of the device that allows a user to expand a distal end of the catheter in an umbrella shape. There is a need for the device's umbrella shaped end to allow fluid flow through the body passage when expanded. There is also a need for the umbrella shape to be retracted such that an object is secured to the distal end of the device while the device is withdrawn from the body passage.

One embodiment of the catheter device has a tube with a proximal end and a distal end. The device also has a linkage inside the tube extending between the proximal end and the distal end of the tube and a plurality of ribs connected to a first connection point at the distal end of the tube. The ribs have a first end at the first connection point and a second end opposite the first end and a mesh connecting to each of the plurality of ribs. The device also has a first handle at the proximal end of the tube and a second handle adjacent to the first handle and attached to the linkage where the second handle actuates the plurality of ribs from a retracted position to generally an extended position from the tube.

Another embodiment of the catheter device has a second handle where the second handle actuates the plurality of ribs by longitudinal movement of the linkage.

Another embodiment of the catheter device has a spring between the first handle and the second handle where the spring is in a loaded condition when the ribs are in the extended position and the spring is in an unloaded condition when the ribs are in the retracted position.

Another embodiment of the catheter device has ribs where at least one rib of the plurality of ribs extends from the tube in an arc.

Another embodiment of the catheter device has a second connection point on the tube proximal to the first connection point where at least one rib of the plurality of ribs extends from the first connection point in the arc to the second connection point on the tube.

Another embodiment of the catheter device has a plurality of ribs where each rib of the plurality of ribs has an atraumatic tip at the second end of each rib.

Another embodiment of the catheter device has an atraumatic tip wherein the atraumatic tip includes a polymer.

Another embodiment of the catheter device has a mesh where the mesh includes a plurality of filaments.

Another embodiment of the catheter device has filaments where the filaments are woven.

Another embodiment of the catheter device has an end cone at the distal end of the tube.

Another embodiment of the catheter device has ribs where each rib has at least one filament connected to the second end of the rib and to the second end of another rib of the plurality of ribs.

Another embodiment of the catheter device has a plurality of ribs where at least one rib of the plurality of ribs includes a shape-memory alloy.

Another embodiment of the catheter device has a plurality of ribs where each rib of the plurality of ribs has a stretcher wire connected at a first end of the stretcher wire to a point on the rib between the first end and second end of the rib and a second end of the stretcher wire connected to the linkage.

Another embodiment of the catheter device has stretcher wires where when the catheter device is in the extended position, at least one stretcher wire presses on at least one rib causing the arc.

Another embodiment of the catheter device has a fluid coupling fitting at the proximal end of the catheter device.

Another embodiment of the catheter device has a tube and linkage where the tube and the linkage are flexible.

An embodiment of the catheter device has a linkage having a proximal end and a distal end and a plurality of ribs connected to a first connection point at the distal end of the linkage. The device has ribs that have a first end at the first connection point and a second end opposite the first end and a mesh connecting to each of the plurality of ribs. The device also has a first handle at the proximal end of the linkage and a second handle adjacent to the first handle and attached to the linkage. The second handle actuates the plurality of ribs from a retracted position to generally an extended position from the linkage.

Another embodiment of the catheter device has linkage where the linkage is flexible.

An embodiment of the catheter device has a tube having a proximal end and a distal end and a linkage inside the tube extending between the proximal end and the distal end of the tube. The device also has a plurality of ribs connected to a first connection point at the distal end of the tube, the ribs have a first end at the first connection point and a second end opposite the first end and a mesh connecting to each of the plurality of ribs. The device also has a first handle at the proximal end of the tube and a second handle adjacent to the first handle and attached to the linkage where the second handle actuates the plurality of ribs from a retracted position to generally an extended position from the tube by longitudinal movement of the linkage. The device also has a spring between the first handle and the second handle, where the spring is in a loaded condition when the ribs are in the extended position and the spring is in an unloaded condition when the ribs are in the retracted position. Each rib of the plurality of ribs has a stretcher wire connected at a first end of the stretcher wire to a point on the rib between the first end and second end of the rib and a second end of the stretcher wire connected to the linkage. At least one rib of the plurality of ribs extends from the shaft in an arc and when the catheter device is in the extended position, at least one stretcher wire presses on at least one rib causing the arc.

An embodiment of the catheter device has a tube having a proximal end and a distal end and a linkage inside the tube extending between the proximal end and the distal end of the tube. The device also has a plurality of ribs connected to a first connection point at the distal end of the tube, the ribs having a first end at the first connection point and a second end opposite the first end and a mesh connecting to each of the plurality of ribs. The device also has an actuator at the proximal end of the tube and connected to the linkage, where the actuator accepts a signal and actuates the plurality of ribs from a retracted position to generally an extended position from the tube.

Another embodiment of the catheter device has an input device connected to the actuator that interacts with and provides a signal to the actuator.

Another embodiment of the catheter device has an input where the input device includes a button.

### BRIEF DESCRIPTION OF THE DRAWINGS

FIG. 1 is an embodiment of the device wherein the capturing portion is in the closed position.

FIG. 2 is an embodiment of the device wherein the net portion of the capturing portion is shown in more detail.

FIG. 3 is an embodiment of the device wherein the capturing portion of the device is in the open position.

FIG. 4 is an embodiment of the device wherein the capturing portion of the device is in the open position and the net of the capturing portion is shown in more detail.

FIG. 5 is an embodiment of the device wherein the capturing portion is in the closed position and there is no tube, just linkage that actuates the ribs.

FIG. 6 is an embodiment of the device wherein the capturing portion is in the closed position and there is an actuator with a button instead of handles.

FIG. 7 is an embodiment of the device wherein the capturing portion is in the closed position and there is an actuator with a signal conduit instead of handles.

FIG. 8 is an embodiment of the device wherein the capturing portion is in the open position forming a basket.

### DETAILED DESCRIPTION

The present teachings are described more fully hereinafter with reference to the accompanying drawings, in which the present embodiments are shown. The following description is presented for illustrative purposes only and the present teachings should not be limited to these embodiments.

In compliance with the statute, the present teachings have been described in language more or less specific as to structural and mechanical features. It is to be understood, however, that the present teachings are not limited to the specific features shown and described, since the apparatus, systems, and methods herein disclosed comprise preferred forms of putting the present teachings into effect.

For purposes of explanation and not limitation, specific details are set forth such as particular structures, architectures, interfaces, techniques, etc. in order to provide a thorough understanding. In other instances, detailed descriptions of well-known devices and methods are omitted so as not to obscure the description with unnecessary detail.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The steps of any method disclosed herein do not have to be performed in the exact order disclosed, unless explicitly stated. The use of “first”, “second,” etc. for different features/components of the present disclosure are only intended to distinguish the features/components from other similar features/components and not to impart any order or hierarchy to the features/components.

To aid the Patent Office and any readers of any patent issued on this application in interpreting the claims appended hereto, Applicant does not intend any of the appended claims or claim elements to invoke 35 U.S.C. 112(f) unless the words “means for” or “step for” are explicitly used in the particular claim.

A notable advantage to the disclosed device is the ability to securely capture an object within a body passage and withdraw the object with the device while only minimally restricting fluid flow through the body passage. Another notable advantage to the disclosed device includes the ability to retrieve obstructive objects while maintaining venous or arterial access via an intraluminal wire extending within the central lumen or flush port of the device. Another notable advantage to the disclosed device includes the variability in potential diameter of the basket portion of the device to adequately oppose large vessel walls or small vessel walls depending on designed and created size of the device. Additionally, a notable advantage to the disclosed device is a self-centering nature of the catheter within a passage lumen due to radial force from the ribs of the device against interior walls of the passage.

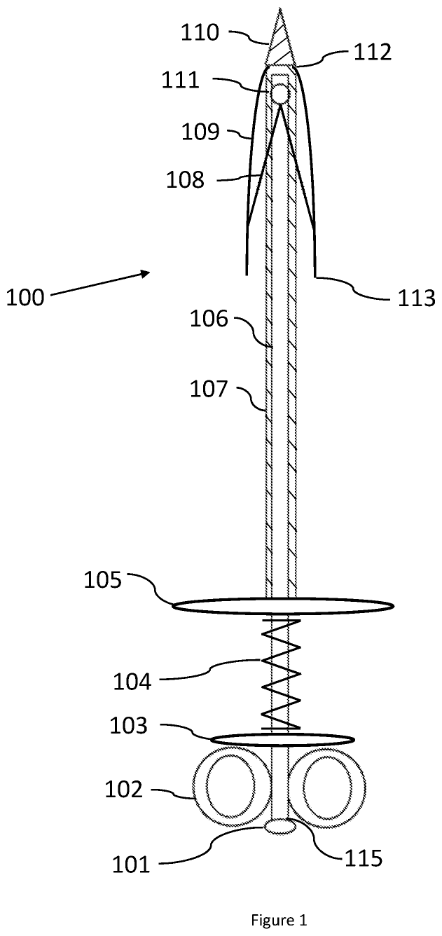

Referring to FIG. 1, one embodiment of the catheter 100 is shown. The catheter 100 can have a tube 107, linkage 106, handle 102, flush and wire port 101, large plastic guard 105, small plastic guard 103, spring 104, ribs 109, retraction wires 108, fixation point 111, a soft end cone 110. The tube 107 is shown in cross sectional view to show an inner passage of the tube 107 containing the linkage 106. The catheter 100 is used to remove or position an object or obstruction within a body passage.

The tube 107 is shaped such that it can be inserted into a body passage including, but not limited to, a vein, artery, urethra, lymph duct, airway, vessel, duct, or any other body passage known in the art. The tube can be flexible, rigid, or have sections of the tube 107 that are rigid and others that are flexible. The diameter and length of the catheter can vary depending on the passage the tube 107 will be inserted into and how far it must be inserted to reach an object. The tube 107 can be manufactured from materials including, but not limited to, polymer, plastic, rubber, metal, composite, or any other material known in the art for constructing medical instruments. Further examples of materials that can be used to construct the tube 107 include, but are not limited to, silicone rubber, polyurethane, polycarbonate, polypropylene, polyethylene, polyester, polystyrene, PVC, polyethersulfone, thermoplastic elastomers, fluoropolymers, Makolon®, Bayfol®, peek, acrylic, stainless steel, titanium, polylactic acid, and any other material known in the art for producing medical instruments and devices.

Further referring to FIG. 1, the tube 107 can contain a linkage or device such that a user can cause the catheter to bend, rotate, or otherwise contort through manipulation of a control. The device can be mechanical, electromechanical, pneumatic, hydraulic, or any other method known in the art for manipulating an instrument. The control for manipulating the catheter's 107 shape can be at a portion of the tube 107 that is outside of the body so that a user can manipulate the control manually, or it can be at a position on the tube 107 inside of the body in the case of an electromechanical or otherwise remote control.

The catheter 100 can have multiple ribs 109 that connect to the tube 107 at a connection area 112. The ribs 109 can move in a hinging motion towards and away from the tube 107. The ribs 109 can form an umbrella shape when in the position generally away from the tube 107. This position can also be called an “extended position.” The hinging motion can be about the connection area 112. The ribs 109 can be rigid, flexible, or have joints connecting lengths of rib 109 and, in the case of a flexible rib 109, it can have a shape memory wherein when a bending force is applied to the rib 109, the rib 109 returns to its unbent shape when the force is removed.

The ribs 109 can be made from materials including, but not limited to, polymer, plastic, rubber, metal, composite, or any other material known in the art for constructing medical instruments. Further examples of materials that can be used to construct the ribs 109 include, but are not limited to, silicone rubber, polyurethane, polycarbonate, polypropylene, polyethylene, polyester, polystyrene, PVC, polyethersulfone, thermoplastic elastomers, fluoropolymers, Makolon®, Bayfol®, peek, acrylic, stainless steel, titanium, polylactic acid, Nitinol®, other shape-memory alloys, and any other material known in the art for producing medical instruments and devices.

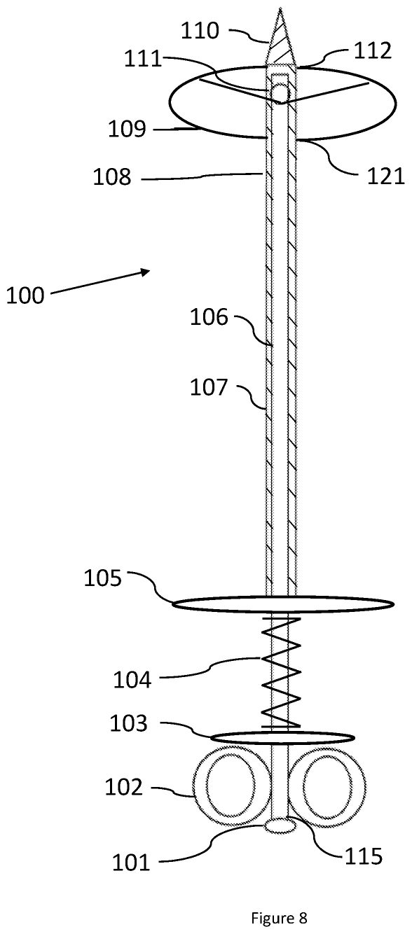

In other embodiments, the ribs 109 can also connect to a second connection point, 121. Actuation of the catheter 100 causes the ribs 109 to form a basket shape as shown in FIG. 8.

The ribs 109 can each have an atraumatic tip 113 on the ends opposite those connected to the tube 107 at connection point 112. The atraumatic tips 113 can be rounded in shape so as to not snag or cut tissue when the device 100 is in use. The atraumatic tips 113 can be made from the same material as the ribs 109 or they can be made from a different material bonded to or otherwise secured to the ends of the ribs 109.

The atraumatic tips 113 can be made from materials including, but not limited to polymer, plastic, rubber, metal, composite, or any other material known in the art for constructing medical instruments. Further examples of materials that can be used to construct the atraumatic tips 113 include, but are not limited to, silicone rubber, polyurethane, polycarbonate, polypropylene, polyethylene, polyester, polystyrene, PVC, polyethersulfone, thermoplastic elastomers, fluoropolymers, Makolon®, Bayfol®, peek, acrylic, stainless steel, titanium, polylactic acid, Nitinol®, and any other material known in the art for producing medical instruments and devices. The atraumatic tips 113 can be bonded using adhesive, epoxy, welding, over molding, molding, pressing, crimping, threading, screws, rivets, pins or any other method known in the art for joining materials together.

The catheter 100 can have an atraumatic end cone 110 at the distal end of the tube 107. The end cone 110 can be a shape including, but not limited to, a cone, truncated cone, pyramid, angled chisel, perpendicular chisel, sphere, elliptic paraboloid, bullet, elliptic hyperparaboloid, or any other shape known in the art for shaping the tip of an instrument for insertion into a body. The atraumatic end cone 110 can be made from materials including, but not limited to, silicone rubber, polyurethane, polycarbonate, polypropylene, polyethylene, polyester, polystyrene, PVC, polyethersulfone, thermoplastic elastomers, fluoropolymers, Makolon®, Bayfol®, peek, acrylic, stainless steel, titanium, polylactic acid, Nitinol®, and any other material known in the art for producing medical instruments and devices. The atraumatic end cone 110 can be bonded to the tube 107 using adhesive, epoxy, welding, over molding, molding, pressing, crimping, threading, screws, rivets, pins or any other method known in the art for joining materials together.

The catheter 100 can have stretchers 108 each connected at one end to the linkage 106, or fixation point 111 and the other end to one rib 109. The stretchers 108 can act on the ribs 109 to move and fix the ribs 109 in relation to the tube 107. The stretchers 108 can be connected to the linkage 106 or the fixation point 111 and the ribs 109 such that they are movable in a hinging motion in relation to the linkage 106 and the ribs 109. The stretchers 108 can be connected to the linkage 106 and the ribs 109 by use of connections including, but not limited to, hinges, flexible joints, adhesives, sealants, moldings, pins, slots, soldering, welding, or any other method known in the art for joining materials. The stretchers 108 can be flexibly or rigidly connected to the linkage 106 or the fixation point 111, and the ribs 109. In the case of a rigid connection, the stretchers 108 can rely on a flexibility of the stretchers 108 for the hinging motion between the stretchers 108 and the linkage 106 and ribs 109.

The stretchers 108 can be made from materials including, but not limited to, Nitinol®, silicone rubber, polyurethane, polycarbonate, polypropylene, polyethylene, polyester, polystyrene, PVC, polyethersulfone, thermoplastic elastomers, fluoropolymers, Makolon®, Bayfol®, peek, acrylic, stainless steel, titanium, polylactic acid, other shape-memory alloys, and any other material known in the art for producing medical instruments and devices.

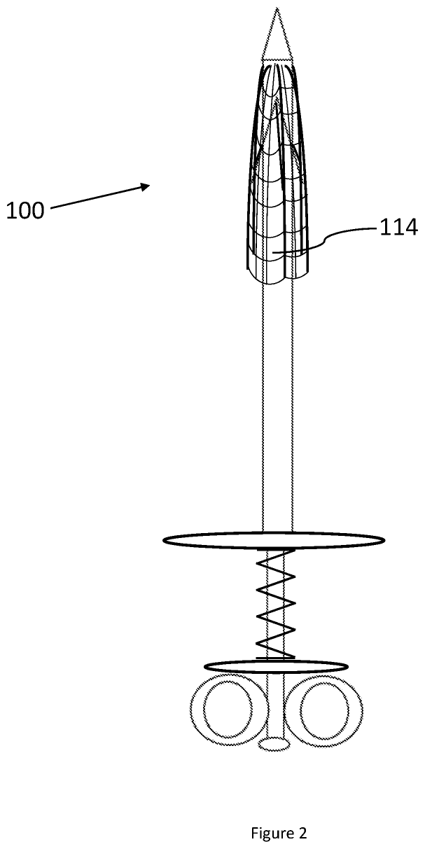

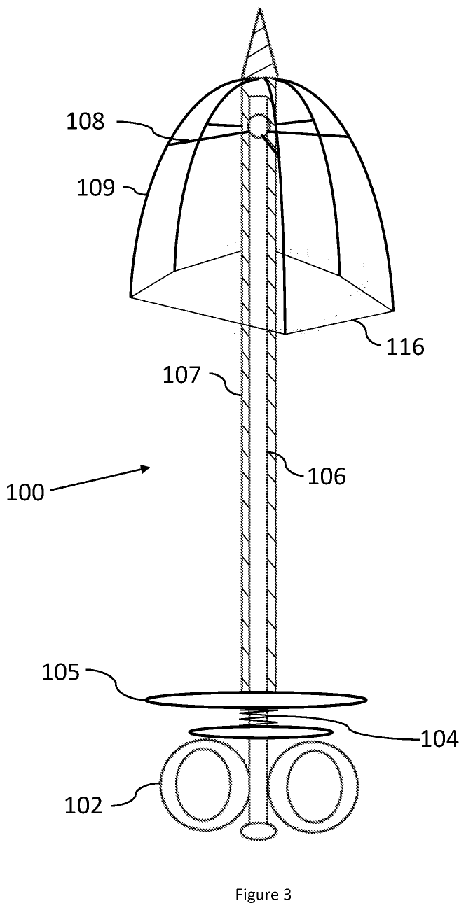

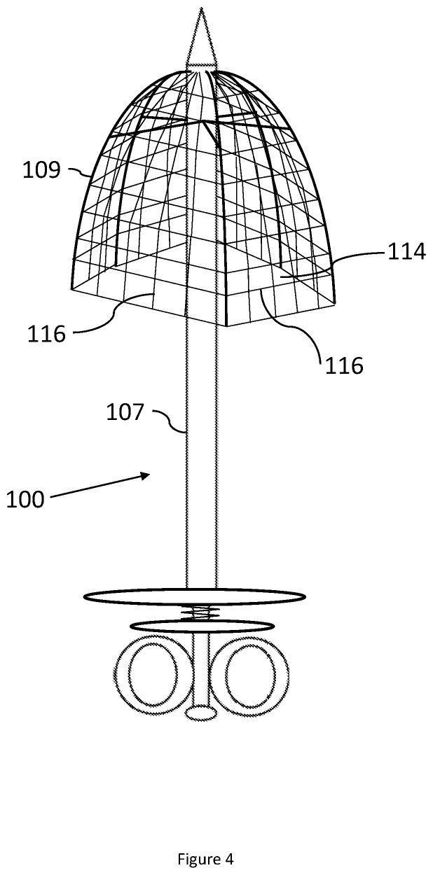

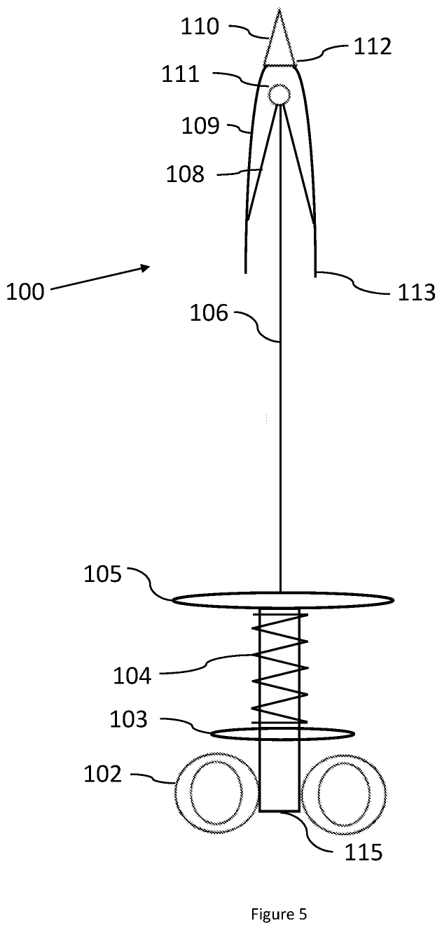

The linkage 106 can be inside of the hollow passage of the tube 107 or, as in the embodiment of FIG. 5, the linkage 106 can connect to the ribs 109 with no tube 107. The linkage 107 can be configured with a fixation point 111 that is connected to the stretchers 108. The linkage 106 can be rigid, flexible, or both, or have joints connecting sections of the linkage 106. The linkage 106 can be hollow or solid and be made up of one piece or multiple pieces. As stated above, the stretchers 108 can be fastened to the linkage 106 or fixation point 111 at the distal end of the tube 107. The linkage 106 can move proximally and distally in relation to the tube 107. In some embodiments, the linkage 106 can have an extended position and a retracted position. In some embodiments, the linkage 106 can be in a proximal position when the catheter 100 is in a retracted position and in others, the linkage 106 can be in a distal position when the catheter 100 is in the retracted position. The retracted position of the device 100 is the position depicted in FIGS. 1 and 2. In some embodiments, the linkage 106 can be in a distal position when the catheter 100 is in an extended position. The extended position of the device 100 is the position depicted in FIGS. 3 and 4. As is illustrated in FIGS. 1 and 2, the ribs 109 of the catheter 100 are held adjacent to the tube 107. As is illustrated in FIGS. 3 and 4, the ribs 109 of the catheter 100 extend away from the tube 107.

The linkage 106 can also move in directions including, but not limited to, rotating clockwise or counter clockwise, or translationally in a direction perpendicular to the long direction of the tube 107, to transition the catheter 100 between the extended and retracted positions. The linkage can be made from materials including, but not limited to, Nitinol®, silicone rubber, polyurethane, polycarbonate, polypropylene, polyethylene, polyester, polystyrene, PVC, polyethersulfone, thermoplastic elastomers, fluoropolymers, Makolon®, Bayfol®, peek, acrylic, stainless steel, titanium, polylactic acid, other shape-memory alloys, and any other material known in the art for producing medical instruments and devices.

When the catheter 100 is in the retracted position, the stretchers 108 can hold the ribs 109 adjacent to the tube 107. The stretchers 108 can hold the ribs 109 in a position by tension, compression, or bending forces.

The catheter 100 can have a plunger 115 at the proximal end of the device 100 that is connected to the linkage 106. The plunger 115 can be shaped such that when the plunger 115 is pressed in the distal direction of the catheter 100, the ribs 109 are actuated away from the catheter 100. The catheter 100 can have a spring 104 that acts to resist movement of the plunger 115 and, upon the removal of distal-direction force on the plunger 115, the spring 104 moving the plunger 115 to a proximal resting position. The proximal resting position of the plunger 115 can correspond with the retracted position of the catheter 100 depicted in FIG. 1. The spring 104 can be made from materials including, plastic, polymer, rubber, metal, elastomer, composite, or any other material known in the art for making springs and medical devices. The spring 104 can be a coil, wave, compressible elastomer sleeve, leaf, or any other design of spring known in the art.

Other embodiments of the catheter 100 can have a plunger 115 shaped such that when the plunger 115 is pressed in the proximal direction of the catheter 100, the ribs 109 are actuated towards the catheter 100. The catheter 100 can have a spring 104 that acts to resist movement of the plunger 115 and, upon removal of the proximal-direction force on the plunger 115, the spring 104 moving the plunger 115 to a distal resting position.

The catheter 100 can have a first handle 102 at the proximal end of the catheter 100. The first handle 102 can be connected to the proximal end of the tube 107. The first handle 102 can have loops through which a user's fingers can pass to gain a secure grip on the catheter 100. The loops can be made of materials including, but not limited to, plastic, rubber, silicone rubber, metal, stainless steel, titanium, polymer, or any other material known in the art for producing medical instruments.

Instead of loops, the first handle 102 can have one or two bars that protrude perpendicular to the tube 107. As a further alternative, the first handle 102 can have a circular or ovular disk through which the tube 107 passes or protrudes from.

The catheter 100 can have a second handle 105 attached to the linkage 106 such that a user grips both the first handle 102 and the second handle 105 and moves the two grips in relation to each other to actuate the catheter 100 from the retracted position to the extended position and vice versa. The second handle 105 can be of the same design or a different design as the first handle 102. The second handle 105 can be any of the designs listed above as options for the first handle 102.

The handles 102 and 105 can be positioned such that a user only needs to use one hand to actuate the catheter 100. For example, a user may hold the handles 102 and 105 such that the user's index finger and middle finger rest on the handle 102 and the user's thumb rests on the handle 105. To actuate the catheter 100, the user presses on the handles 102 and 105 with the above referenced fingers, bringing their fingers together in a grasping motion. This motion brings the handles 102 and 105 together and actuates the catheter 100.

Alternatively, the first handle 102 can be attached to the proximal end of the linkage 106 and operate as the plunger 115 is described above. The second handle 105 can be attached to the tube 107. This embodiment is described in FIGS. 1-4. Regardless of the handle 102 and 105 configuration, the handles can be positioned such that a user can use fingers from one hand to move the handles 102 and 105. Such configuration can allow the user to actuate the catheter 100 with one hand.

The tube 107 or linkage 106 can have a fluid fitting 103 at or near the proximal end. The fluid fitting 103 can be connectable to a fluid conduit that allows fluid flow to or from a body cavity or passage. The fluid fitting 103 can be connectable to a fluid conduit that supplies fluid flow for injection into or suction from a body cavity. The fluid flow can be supplied to flush material from the tube 107 or the distal end of the catheter 100. The fluid fitting 103 can include, but is not limited to, a luer lock fitting, threaded fitting, quick connect fitting, tapered fitting, barb fitting, bayonet fitting, or any other fitting known in the art for making a connection with a fluid conduit.

The fluid fitting 103 or the tube 107 can include a valve for controlling fluid through the fluid fitting 103. The valve can be a design including, ball valve, poppet valve, spool valve, gate valve, tube clamp, or any other valve known in the art for control of fluid flow through a fluid conduit.

Referring to FIG. 2, the catheter 100 in the retracted position is shown in more detail. The catheter 100 can have a mesh 114 extending between the ribs 109. The mesh 114 can connect to each of the ribs 109. The mesh 114 can be made up of filaments, strings, cords, strands, threads, woven threads, or fabric (hereinafter referred to as “filaments”) extending from each rib 109 to each next adjacent rib 109 or any other rib 109. The filaments can be made to be flexible and/or woven. The filaments can be made from materials including, but not limited to, Nitinol®, silicone rubber, polyurethane, polycarbonate, polypropylene, polyethylene, polyester, polystyrene, PVC, polyethersulfone, thermoplastic elastomers, fluoropolymers, Makolon®, Bayfol®, peek, acrylic, stainless steel, titanium, polylactic acid, other shape-memory alloys, and any other material known in the art for producing flexible filaments.

The mesh 114 can be made up of filaments that contract or bend when the catheter 100 is transitioned into the retracted position. The mesh 114 can contract to be adjacent to the tube 107 or can contract to be inside of the tube 107. The mesh 114 can be partially made up of rigid filaments and partially of flexible filaments. The ratio of rigid to flexible filaments can change based upon the desired retracted and extended shapes of the rib 109 and mesh 114 assembly.

Referring to FIG. 3, an embodiment of the catheter 100 is shown in the extended position without the mesh 114 for clarity. In the extended position, the ribs 109 can extend away from the tube 107. The ribs 109 can be actuated to and held in the extended position by the stretchers 108. The stretchers 108 can apply a force to the ribs 109 such that the ribs form an arc, the plurality of arc-shaped ribs forming an umbrella shape as shown. The ribs 109 can have elongated elements 116 at the distal ends of the ribs 109 that connect to the distal ends of the next adjacent ribs 109. These elongated elements 116 can secure the distal ends of the ribs 109 to each other such that a particular shape is made by the ends of the ribs 109 in the extended position. For example, from the proximal end point of view and the catheter 100 in the extended position, the distal ends of the ribs can appear to create a hexagonal shape if all of the elongated elements 116 are of equal length and there are 6 ribs 109. Alternatively, an elongated, six-sided polygon can be formed if some of the elongated elements 116 are of a shorter length than others. The catheter 100 shape can be customized by changing the length of the elongated elements 116 at the distal ends of the ribs 109 and the number of ribs 109.

The catheter 100 shape can have a diameter across an outer perimeter of the extended ribs 109. This diameter can vary to adequately oppose the inner surfaces of a variety of sizes of vessels including large vessel walls and small vessel walls. The diameter of the catheter 100 shape can vary based upon the structure of the ribs 109 as designed for manufacture. The diameter, length, cross section, and material of each rib 109 can be selected to provide a specific catheter 100 shape.

When in the extended position, the catheter 100 can be centered within a passage by the extension of the ribs 109 and their contact with an interior surface of the passage. The ribs 109 can extend in way such that the catheter has a self-centering characteristic by which each of the ribs 109 extend equally from the tube 107.

The handles 102 and 105 are shown in their actuated positions and the spring 104 in a loaded condition. In the embodiment shown, the resting position of the catheter 100 is the retracted position. The handles 102 and 105 have been pressed so they are closer together, in effect, moving the linkage 106 in the distal direction and actuating the ribs of the catheter 100. In other embodiments, the handles 102 and 105 could be rotated in relation to each other to actuate the catheter 100, or the handles 102 and 105 could be separated from each other to actuate the catheter 100. In other embodiments, the spring 104 could be put into an unloaded position when the catheter 100 is actuated to an extended position making it so the catheter 100 tends towards the extended position.

Referring to FIG. 4, an embodiment of the catheter 100 in the extended position is shown with the mesh 114. Elongated elements 116 making the mesh 114 are shown extending from the ribs 109 to the next adjacent ribs 109 on the tube 107. The elongated elements 116 can be woven with each other or fused to each other to form the mesh 114. The elongated elements 116 can also connect to a rib 109 to another elongated element to form a mesh 114. The mesh 114 can have gaps through which fluids and objects under a certain size can pass such as blood or cells. The mesh 114 can be sized such that certain sized or shaped objects are captured by the mesh 114. The size of the mesh 114 can be specified by using different numbers of elongated elements 116 and positioning them in different woven or overlapping configurations.

Additionally, the mesh 114 can be made from planar material having perforations or holes such that fluid can pass through the mesh while an object is captured. The mesh can be made without elongated elements.

An intraluminal wire can extend within the tube 107, the tube 107 providing access to a passage including a vein or an artery. The intraluminal wire can be used within the tube 107 or the flush port 101 to retrieve obstructive objects within the passage.

Referring to FIG. 5, another embodiment of the catheter 100 can have a linkage 106 that has the ribs 109 connected to the linkage's 106 distal end. Moving the handle 102 towards handle 105 can actuate the ribs 109 away from the linkage 106. The ribs 109 can be actuated to a generally extended position from the linkage 106. In other embodiments, other handle 102 and 105 movements described above can actuate the ribs 109 of the catheter 100.

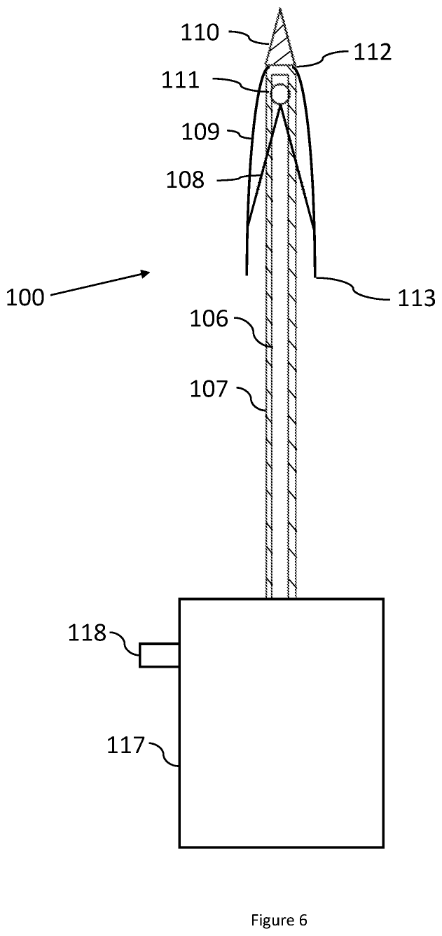

Referring to FIG. 6, the catheter 100 can have an actuator 117 instead of handles 102 and 105. The actuator 117 can have a button 118 with which the user creates a signal to indicate to the actuator 117 to actuate the catheter 100. The actuator 117 can be connected to the linkage 106 and move the linkage 106 to actuate the catheter 100. The actuator 117 can include, piezoelectric devices, solenoids, stepper motors, linear motors, inductance devices, magnetic devices, hydraulic devices, pneumatic devices, or any other device for creating mechanical motion from a signal. The actuator 117 can have multiple buttons 118. Button 118 can include push buttons, switches, momentary switches or buttons, slider switches, rheostats, touch sensors, heat sensors, capacitance sensors, or any other device known in the art for detecting a user's interface with a device.

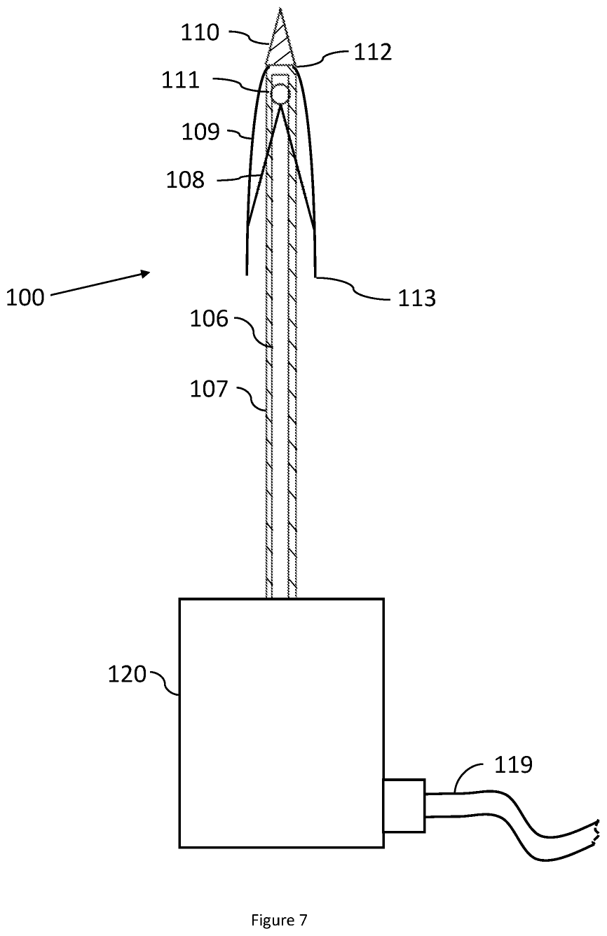

Referring to FIG. 7, the catheter 100 can have a control device 120 that accepts a signal through a conduit 119. The control device 120 can actuate the catheter 100 upon receipt of a signal through conduit 119. The control device 120 can include the devices of the actuator 117 and other mechanical devices that can create mechanical motion. The conduit 119 can include a wire, tube, pipe, fiber, or any other conduit known in the art for conducting a signal.

Referring to FIG. 8, the catheter 100 can have ribs 109 that when the catheter 100 is actuated, the ribs 109 form a disk or basket shape perpendicular to the tube 107. The ribs can form the disk or basket shape by connecting to the tube 107 both the connection point 112 at one end and the second connection point at 121. The ribs 109 can have a mesh 114 connected to the ribs 109 that forms the surfaces of the disk or basket shape. The stretcher wires 108 can act on the ribs 109 to actuate the catheter 100.

An exemplary embodiment of the disclosed apparatus is described below.

The catheter 100 is made of a tube 107, linkage 106, ribs 109, stretchers 108, a mesh 114 between the ribs 109, and handles 102 and 105. A guidewire or similar device is inserted in to a vein in a patient to a location of an object. The catheter 100 is inserted into the vein and past an object in the vein in a human patient over the guidewire. The handles 102 and 105 are shaped such that a surgeon can hold them between their thumb, first (index), and second (middle) fingers. The surgeon's thumb rests on the handle 102 that is attached to the linkage 106 and the surgeons first and second fingers rest on the second handle which is attached to the tube 107. In some cases, a guide wire is not necessary to guide the catheter 100 and not used. In those cases, the catheter 100 is inserted directly into a passage without the guidewire.

The surgeon actuates the catheter 100 inside of the vein by pressing their thumb against the handle 102, moving the handle 102 towards the handle 105, in effect moving the linkage 106. The linkage 106 has ends of stretchers 108 connected to it. The linkage 106 moves the ends of the stretchers 108 in relation to the tube 107 in effect moving the rest of the stretchers 108 away from the tube 107 in a spreading motion. Each stretcher wire 108 acts on a rib 109 and spreads the ribs 109 from the tube 107 forming the frame of an umbrella shape.

The ribs 109 have elongated elements 116 connected between them forming a mesh 114. The mesh 114 is made taunt by the extension of the ribs 109. The surgeon uses the extended ribs 109 and the mesh 114 to capture the object in the vein by holding the catheter 100 in the extended position and partially withdrawing the catheter 100 from the vein. The surgeon can then release one of the handles 102 or 105, to allow the catheter 100 to transition to the retracted position. In the retracted position, the handles 102 and 105 separate from each other and the linkage 106 moves in relation to the tube 107. The stretchers 108 and ribs 109 are retracted towards the tube 107 and the ribs 109 are retracted towards the tube 107. The mesh 114 is closed around the object, securing the object to the distal end of the catheter 100. The surgeon then fully withdraws the catheter 100 from the vein with the object securely attached to the catheter 100.

Atraumatic tips on the distal tips of the ribs 109 and the distal tip of the tube 107 prevent damage to the walls of the vein. The atraumatic tips also allow the catheter 100 to move more freely through the vein and past an object in the vein.

Once withdrawn from the vein, the catheter 100 can be transitioned to the extended position to release the object for disposal or examination. The catheter 100 can then be disposed of or sterilized for reuse.

What is claimed is:

1. A catheter device, comprising:
a tube having a proximal end and a distal end;
a linkage inside the tube extending between the proximal end and the distal end of the tube;
a plurality of ribs connected to a first connection point at the distal end of the tube, the ribs having a first end at the first connection point and a second end opposite the first end;
a mesh connecting to each of the plurality of ribs;
a first handle at the proximal end of the tube;
a second handle adjacent to the first handle and attached to the linkage;
wherein the second handle actuates the plurality of ribs from a retracted position to generally an extended position from the tube.

2. The catheter device of claim 1, wherein:
the second handle actuates the plurality of ribs by longitudinal movement of the linkage.

3. The catheter device of claim 1, further comprising:
a spring between the first handle and the second handle;
wherein the spring is in a loaded condition when the ribs are in the extended position and the spring is in an unloaded condition when the ribs are in the retracted position.

4. The catheter device of claim 1,
wherein at least one rib of the plurality of ribs extends from the tube in an arc.

5. The catheter device of claim 4, further comprising:
a second connection point on the tube proximal to the first connection point;
wherein at least one rib of the plurality of ribs extends from the first connection point in the arc to the second connection point on the tube.

6. The catheter device of claim 1, further comprising:
each rib of the plurality of ribs having an atraumatic tip at the second end of each rib.

7. The catheter device of claim 1, wherein the mesh comprises a plurality of filaments.

8. The catheter device of claim 7, wherein the filaments are woven.

9. The catheter device of claim 1, further comprising an end cone at the distal end of the tube.

10. The catheter device of claim 1, further comprising:
each rib having at least one filament connected to the second end of the rib and to the second end of another rib of the plurality of ribs.

11. The catheter device of claim 4, wherein at least one rib of the plurality of ribs comprises shape-memory alloy.

12. The catheter device of claim 4, further comprising:
each rib of the plurality of ribs having a stretcher wire connected at a first end of the stretcher wire to a point on the rib between the first end and second end of the rib;
a second end of the stretcher wire connected to the linkage.

13. The catheter device of claim 12, wherein when the catheter device is in the extended position, at least one stretcher wire presses on at least one rib causing the arc.

14. The catheter device of claim 1, further comprising:
a fluid coupling fitting at the proximal end of the catheter device.

15. The catheter device of claim 1, wherein the tube and the linkage are flexible.

16. A catheter device, comprising:
a linkage having a proximal end and a distal end;
a plurality of ribs connected to a first connection point at the distal end of the linkage, the ribs having a first end at the first connection point and a second end opposite the first end;
a mesh connecting to each of the plurality of ribs;
a first handle at the proximal end of the linkage;
a second handle adjacent to the first handle and attached to the linkage;
wherein the second handle actuates the plurality of ribs from a retracted position to generally an extended position from the linkage.

17. The catheter device of claim 16, wherein the linkage is flexible.

18. A catheter device, comprising:
a tube having a proximal end and a distal end;
a linkage inside the tube extending between the proximal end and the distal end of the tube;
a plurality of ribs connected to a first connection point at the distal end of the tube, the ribs having a first end at the first connection point and a second end opposite the first end;
a mesh connecting to each of the plurality of ribs;
an actuator at the proximal end of the tube and connected to the linkage;
wherein the actuator accepts a signal and actuates the plurality of ribs from a retracted position to generally an extended position from the tube.

19. The catheter device of claim 18 further comprising:
an input device connected to the actuator that interacts with and provides a signal to the actuator.

20. The catheter device of claim 19, wherein the input device comprises a button.
